(19) United States
(12) Patent Application Publication
(10) Pub. No.: US 2022/0280574 A1
(43) Pub. Date: Sep. 8, 2022

(54) THERAPEUTIC MONOCYTES FOR PREVENTION OF SUICIDAL IDEATION

(71) Applicant: Therapeutic Solutions International, Inc, Oceanside, CA (US)

(72) Inventors: Thomas E. Ichim, Oceanside, CA (US); Timothy G. Dixon, Oceanside, CA (US); Famela Ramos, Oceanside, CA (US); Kalina O'Connor, Oceanside, CA (US); James Veltmeyer, Oceanside, CA (US)

(73) Assignee: Therapeutic Solutions International, Inc., OCEANSIDE, CA (US)

(21) Appl. No.: 17/687,478

(22) Filed: Mar. 4, 2022

Related U.S. Application Data
(60) Provisional application No. 63/156,850, filed on Mar. 4, 2021.

(51) Int. Cl. A61K 35/51; A61K 35/16; A61P 25/24; A61K 35/19

(52) U.S. Cl. CPC A61K 35/51; A61K 35/16; A61P 25/24; A61K 35/19

(57) ABSTRACT

The invention discloses compositions of matter, protocols, and therapeutic means for treatment of suicidal ideations and/or suppression of suicidal attempts. In one embodiment the invention provides the use of umbilical cord derived monocytes as a means of treatment. In another embodiment, monocytes are de-differentiated from adult monocytes using reprogramming means to create monocyte capable of producing anti-inflammatory as well as regenerative properties useful in reducing suicidal ideations and/or attempts.

### CROSS-REFERENCE TO RELATED APPLICATIONS

This application claims the benefit of priority to U.S. Provisional Application No. 63/156,850, filed Mar. 4, 2021, the contents of which are incorporated herein by reference.

### FIELD OF THE INVENTION

The invention pertains to the field of suicide prevention, and more specifically the use of monocytes to prevent suicidal ideation and suicide

### BACKGROUND

There is a need in the art for improved methods and therapeutics for preventing suicidal ideation and suicide.

### SUMMARY

Preferred embodiments include methods of inhibiting suicidal ideations, and/or propensity towards suicide through administration umbilical cord blood mononuclear cells.

Preferred methods include embodiments wherein said umbilical cord blood mononuclear cells are allogeneic.

Preferred methods include embodiments wherein said umbilical cord blood mononuclear cells are autologous.

Preferred methods include embodiments wherein said umbilical cord blood mononuclear cells are xenogeneic.

Preferred methods include embodiments wherein said umbilical cord blood mononuclear cells are CD34 cells.

Preferred methods include embodiments wherein said umbilical cord blood mononuclear cells are CD133 cells.

Preferred methods include embodiments wherein said umbilical cord blood mononuclear cells are VEGFR-2 expressing cells.

Preferred methods include embodiments wherein said umbilical cord blood mononuclear cells are hematopoietic stem cells.

Preferred methods include embodiments wherein said umbilical cord blood mononuclear cells are endothelial progenitor cells.

Preferred methods include embodiments wherein said umbilical cord blood mononuclear cells are plastic adherent.

Preferred methods include embodiments wherein said umbilical cord blood mononuclear cells are monocytes.

Preferred methods include embodiments wherein said monocytes are M2 monocytes.

Preferred methods include embodiments wherein said M2 monocytes are administered together with platelet rich plasma, wherein said cells together comprise a composition suitable for injection into an individual, and wherein said M2 monocytes are OCT-4 positive as determinable by RT-PCR, are adherent to tissue culture plastic, and are not trophoblasts.

Preferred methods include embodiments wherein injection of said composition to said individual results in prolonged localization of said M2 cells at the site of injection, relative to M2 cells not combined with platelet rich plasma.

Preferred methods include embodiments wherein said platelet rich plasma is autologous platelet rich plasma.

Preferred methods include embodiments wherein said platelet rich plasma is derived from placental perfusate.

Preferred methods include embodiments wherein the volume to volume ratio of M2 cells to platelet rich plasma in the composition is between about 10:1 and 1:10.

Preferred methods include embodiments wherein the volume to volume ratio of M2 cells to platelet rich plasma in the composition is about 1:1.

Preferred methods include embodiments wherein the ratio of the number of M2 cells to the number of platelets in the platelet rich plasma is between about 100:1 and 1:100.

Preferred methods include embodiments wherein the ratio of the number of M2 cells to the number of platelets in the platelet rich plasma is about 1:1.

Preferred methods include embodiments wherein said M2 cells are CD49f positive.

Preferred methods include embodiments wherein said M2 cells are CD105 positive.

Preferred methods include embodiments wherein said M2 cells are CD200 positive.

Preferred methods include embodiments wherein said M2 cells are VEGFR1/Flt-1 positive.

Preferred methods include embodiments wherein said M2 cells are VEGFR2/KDR positive.

Preferred methods include embodiments wherein said M2 cells are CD90 positive.

Preferred methods include embodiments wherein said M2 cells are OCT4 positive, HLA-G positive, and CD90 positive.

Preferred methods include embodiments wherein said M2 cells are CD9 positive.

Preferred methods include embodiments wherein said M2 cells are CD10 positive.

Preferred methods include embodiments wherein said M2 cells are CD44 positive.

Preferred methods include embodiments wherein said M2 cells are CD54 positive.

Preferred methods include embodiments wherein said M2 cells are CD98 positive.

Preferred methods include embodiments wherein said M2 cells are CD98 positive and Tie2 positive.

Preferred methods include embodiments wherein said M2 cells are TEM7 positive.

Preferred methods include embodiments wherein said M2 cells are CD31 positive and CD34 positive.

Preferred methods include embodiments wherein said M2 cells are CXCR4 positive.

Preferred methods include embodiments wherein said M2 cells express mRNA encoding for ACTA2.

Preferred methods include embodiments wherein said M2 cells express mRNA encoding for ADAMTS1.

Preferred methods include embodiments wherein said M2 cells express mRNA encoding for ANG.

Preferred methods include embodiments wherein said M2 cells express mRNA encoding for ANGPT1.

Preferred methods include embodiments wherein said M2 cells express mRNA encoding for ANGPT2.

Preferred methods include embodiments wherein said M2 cells express mRNA encoding for ANGPTL1.

Preferred methods include embodiments wherein said M2 cells express mRNA encoding for ANGPTL4.

Preferred methods include embodiments wherein said M2 cells express mRNA encoding for BAH.

Preferred methods include embodiments wherein said M2 cells express mRNA encoding for CD44.

Preferred methods include embodiments wherein said M2 cells express mRNA encoding for CD200.

Preferred methods include embodiments wherein said M2 cells express mRNA encoding for CEACAM1.

Preferred methods include embodiments wherein said M2 cells express mRNA encoding for CHGA.

Preferred methods include embodiments, wherein said M2 cells express mRNA encoding for COL15A1.

Preferred methods include embodiments wherein said M2 cells express mRNA encoding for COL18A1.

Preferred methods include embodiments wherein said M2 cells express mRNA encoding for COL4A1.

Preferred methods include embodiments wherein said M2 cells express mRNA encoding for COL4A2.

Preferred methods include embodiments wherein said M2 cells express mRNA encoding for CSF3.

Preferred methods include embodiments wherein said M2 cells express mRNA encoding for CTGF.

Preferred methods include embodiments wherein said M2 cells express mRNA encoding for CXCL12.

Preferred methods include embodiments wherein said M2 cells express mRNA encoding for CXCL2.

Preferred methods include embodiments wherein said M2 cells express mRNA encoding for DNMT3B.

Preferred methods include embodiments wherein said M2 cells express mRNA encoding for ECGF1.

Preferred methods include embodiments wherein said M2 cells express mRNA encoding for EDG1.

Preferred methods include embodiments wherein said M2 cells express mRNA encoding for EDIL3.

Preferred methods include embodiments wherein said M2 cells express mRNA encoding for ENPP2.

Preferred methods include embodiments wherein said M2 cells express mRNA encoding for EPHB2.

Preferred methods include embodiments wherein said M2 cells express mRNA encoding for FBLN5.

Preferred methods include embodiments wherein said M2 cells express mRNA encoding for F2.

Preferred methods include embodiments wherein said M2 cells express mRNA encoding for FGF1.

Preferred methods include embodiments wherein said M2 cells express mRNA encoding for FGF2.

Preferred methods include embodiments wherein said M2 cells express mRNA encoding for FIGF.

Preferred methods include embodiments wherein said M2 cells express mRNA encoding for FLT4.

Preferred methods include embodiments wherein said M2 cells express mRNA encoding for FST.

Preferred methods include embodiments wherein said M2 cells express mRNA encoding for FOXC2.

Preferred methods include embodiments wherein said M2 cells express mRNA encoding for GRN.

Preferred methods include embodiments wherein said M2 cells express mRNA encoding for HGF.

Preferred methods include embodiments wherein said M2 cells express mRNA encoding for HEY1.

Preferred methods include embodiments wherein said M2 cells express mRNA encoding for HSPG2.

Preferred methods include embodiments wherein said M2 cells express mRNA encoding for IFNB1.

Preferred methods include embodiments wherein said M2 cells express mRNA encoding for IL8.

Preferred methods include embodiments wherein said M2 cells express mRNA encoding for IL12A.

Preferred methods include embodiments wherein said M2 cells express mRNA encoding for ITGA4.

Preferred methods include embodiments wherein said M2 cells express mRNA encoding for ITGAV.

Preferred methods include embodiments wherein said M2 cells express mRNA encoding for ITGB 3.

Preferred methods include embodiments wherein said M2 cells express mRNA encoding for MDK.

Preferred methods include embodiments wherein said M2 cells express mRNA encoding for MMP2.

Preferred methods include embodiments wherein said M2 cells express mRNA encoding for NRP1.

Preferred methods include embodiments wherein said M2 cells express mRNA encoding for NRP2.

Preferred methods include embodiments wherein said M2 cells express mRNA encoding for PDGFB.

Preferred methods include embodiments wherein said M2 cells express mRNA encoding for PDGFRA.

Preferred methods include embodiments wherein said M2 cells express mRNA encoding for PDGFRB.

Preferred methods include embodiments wherein said M2 cells express mRNA encoding for PECAM1.

Preferred methods include embodiments wherein said M2 cells express mRNA encoding for PF4.

Preferred methods include embodiments wherein said M2 cells express mRNA encoding for PGK1.

Preferred methods include embodiments wherein said M2 cells express mRNA encoding for PROX1.

Preferred methods include embodiments wherein said M2 cells express mRNA encoding for PTN.

Preferred methods include embodiments wherein said M2 cells express mRNA encoding for SEMA3F.

Preferred methods include embodiments wherein said M2 cells express mRNA encoding for SERPINB5.

Preferred methods include embodiments wherein said M2 cells express mRNA encoding for SERPINC1.

Preferred methods include embodiments wherein said M2 cells express mRNA encoding for SERPINF1.

Preferred methods include embodiments wherein said M2 cells express mRNA encoding for TIMP3.

Preferred methods include embodiments wherein said M2 cells express mRNA encoding for TGFA.

Preferred methods include embodiments wherein said M2 cells express mRNA encoding for TGFB1.

Preferred methods include embodiments wherein said M2 cells express mRNA encoding for THBS1.

Preferred methods include embodiments wherein said M2 cells express mRNA encoding for THBS2.

Preferred methods include embodiments wherein said M2 cells express mRNA encoding for TIE1.

Preferred methods include embodiments wherein said M2 cells express mRNA encoding for VASH1.

Preferred methods include embodiments wherein said M2 cells synethesize one or more of the proteins selected from a group comprising of Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A; secrete vascular endothelial growth factor (VEGF), hepatocyte growth factor (HGF), interleukin-8 (IL-8), monocyte chemotactic protein-3 (MCP-3), FGF2, Follistatin, G-CSF, EGF, ENA-78, GRO, IL-6, MCP-1, PDGF-BB, TIMP-2, uPAR, or galectin-3

Preferred methods include embodiments wherein cord blood derived monocytes are treated with oxytocin at a concentration of 0.001 IU/million cells to 10 IU/million cells prior to administration.

Preferred methods include embodiments of inducing cessation of neuroinflammation through administration of cord blood mononuclear cells of claim 1.

Preferred methods include embodiments of inducing cessation of cocaine addiction through administration of cord blood mononuclear cells of claim 1.

Preferred methods include embodiments or inducing cessation of opioid addiction through administration of cord blood mononuclear cells of claim 1.

Preferred methods include embodiments or inducing cessation of major depressive disorder through administration of cord blood mononuclear cells of claim 1.

v or inducing cessation of PTSD through administration of cord blood mononuclear cells of claim 1.

Preferred methods include embodiments or inducing cessation of schizophrenia through administration of cord blood mononuclear cells of claim 1.

Preferred methods include embodiments or inducing cessation of mental illness through administration of cord blood mononuclear cells of claim 1.

113. The method of claim 1, wherein said cord blood mononuclear cells are differentiated into myeloid suppressor cells.

Preferred methods include embodiments wherein said cord blood mononuclear cells are differentiated into immature dendritic cells.

Preferred methods include embodiments of inhibiting, or inducing cessation of SAR-CoV-2 associated neuroinflammation/neurodegeneration.

### BRIEF DESCRIPTION OF THE DRAWINGS

FIG. 1 is a bar graph showing suppression of inflammation induced memory dysfunction by cord blood derived monocytes.

FIG. 2 is a bar graph showing suppression of LPS induced plasma IL-6 by cord blood derived monocytes

### DESCRIPTION OF THE INVENTION

The invention provides means of reducing suicidal ideations and/or suicide attempts using compositions comprising type 2 monocytes combined with platelet rich plasma, wherein administration of the compositions to an individual in need thereof results in prolonged localization of the type 2 monocytes at the site of injection or implantation, relative to administration of type 2 monocytes not combined with platelet rich plasma. Amount of type 2 monocytes administered, as well as frequency, may be determined by psychological evaluation, and/or by evaluation of inflammatory cytokines in the blood and/or the cerebral spinal fluid. The administration of other cells together with type 2 monocytes is a also described in the current invention.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Modified Germ Cell: As used herein, “modified germ cell” refers to a cell comprised of a host enucleated ovum and a donor nucleus from a spermatogonia, oogonia or a primordial sex cell. The host enucleated ovum and donor nucleus can be from the same or different species. A modified germ cell can also be called a “hybrid germ cell.”

Multipotent: As used herein, “multipotent” refers to cells that can give rise to several other cell types, but those cell types are limited in number. An example of a multipotent cells is hematopoietic cells—blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells. Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm.

In certain embodiments, the Type 2 monocytes are human. In other embodiments, the platelet rich plasma is human, e.g., is obtained from or derived from a human source. In other embodiments, both the Type 2 monocytes and PRP are human. In various embodiments, the volume to volume ratio of Type 2 monocytes (e.g., Type 2 monocytes in suspension) to platelet rich plasma can be between about 10:1 and 1:10. In some embodiments, the volume to volume ratio of Type 2 monocytes to platelet rich plasma is about 10:1, 9.5:1, 9:1, 8.5:1, 8:1, 7.5:1, 7:1, 6.5:1, 6:1, 5.5:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1.9.5, or 1:10. In particular embodiments, the volume to volume ratio of Type 2 monocytes to platelet rich plasma is about 1:1. In some embodiments, the ratio of the number of Type 2 monocytes to the number of platelets in the platelet rich plasma can be between about 100:1 and 1:100. In some embodiments, the volume to volume ratio of Type 2 monocytes to platelet rich plasma is about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10 1:15, 1:20, 1:25, 1:30, 1:35, 1:40, 1:45, 1:50, 1:55, 1:60, 1:65, 1:70, 1:75, 1:80, 1:85, 1:90, 1:95, or 1:100. In particular embodiments, the ratio of the number of Type 2 monocytes to the number of platelets in the platelet rich plasma is about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10 1:15, 1:20, 1:25, 1:30, 1:35, 1:40, 1:45 1:50, 1:55, 1:60, 1:65, 1:70, 1:75, 1:80, 1:85, 1:90, 1:95, or 1:100.

The compositions comprising Type 2 monocytes and platelet rich plasma provided herein can comprise a therapeutically-effective amount of Type 2 monocytes, platelets, e.g., platelet rich plasma, or both. The combination compositions can comprise at least 1.times.10.sup.4, 5.times.10.sup.4, 1.times.10.sup.5, 5.times.10.sup.5, 1.times.10.sup.6, 5.times.10.sup.6, 1.times.10.sup.7, 5.times.10.sup.7, 1.times.10.sup.8, 5.times.10.sup.8, 1.times.10.sup.9, 5.times.10.sup.9, 1.times.10.sup.10, 5.times.10.sup.10, or 1.times.10.sup.11 Type 2 monocytes, platelets in platelet rich plasma, or both, or no more than 1.times.10.sup.4, 5.times.10.sup.4, 1.times.10.sup.5, 5.times.10.sup.5, 1.times.10.sup.6, 5.times.10.sup.6, 1.times.10.sup.7, 5.times.10.sup.7, 1.times.10.sup.8, 5.times.10.sup.8, 1.times.10.sup.9, 5.times.10.sup.9, 1.times.10.sup.10, 5.times.10.sup.10, or 1.times.10.sup.11 Type 2 monocytes, platelets in platelet rich plasma, or both.

The compositions and methods provided herein use Type 2 monocytes in combination with platelet rich plasma (PRP). In some embodiments, PRP useful in the combination compositions and methods provided herein comprises platelet cells at a concentration of at least 1.1-fold greater than the concentration of platelets in whole blood, e.g., unprocessed whole blood. In some embodiments, the PRP comprises platelet cells at a concentration of about 1.1-fold to about 10-fold greater than the concentration of platelets in whole blood. In some embodiments, the PRP comprises platelet cells at a concentration of about 1.5, 2.0, 2.5, 3.0, 3.5, 4, 4.5, 5, 5.5, 6, 6.5, 7, 7.5, 8, 8.5, 9, 9.5, 10-fold, or more than 10-fold greater than the concentration of platelets in whole blood. Generally, a microliter of whole blood comprises between 140,000 and 500,000 platelets. In some embodiments, the platelet concentration in the PRP useful in the combination compositions and methods provided herein is between about 150,000 and about 2,000,000 platelets per microliter. In some embodiments, the platelet concentration in the PRP is about 150,000, 200,000, 300,000, 400,000, 500,000, 600,000, 700,000, 800,000, 900,000, 1,000,000, 1,100,000, 1,100,000, 1,200,000, 1,300,000, 1,400,000, 1,500,000, 1,600,000, 1,700,000, 1,800,000, 1,900,000, or 2,000,000 platelets per microliter. In some embodiments, the platelet concentration in the PRP is about 2,500,000 to about 5,000,000, or about 5,000,000 to about 7,000,000 platelets per microliter. The combination compositions provided herein may comprise PRP derived from a human or animal source of whole blood. The PRP may be prepared from an autologous source, an allogeneic source, a single source, or a pooled source of platelets and/or plasma, e.g., platelets harvested from placental perfusate. The PRP can be isolated from whole blood or portions of whole blood using a variety of techniques comprising, for example, centrifugation, gravity filtration, and/or direct cell sorting.

It is known that PRP can be, e.g., prepared from a donor who has not been previously treated with a thrombolytic agent, such as heparin, tPA, or aspirin. In some embodiments, the donor has not received a thrombolytic agent for at least 2 hours, 1 day, 2 weeks, or 1 month prior to withdrawing the blood for extraction of the PRP.

In order to derive PRP from donor blood, whole blood may be collected from the donor, for example, using a blood collection syringe. The amount of blood collected may depend on a number of factors, including, for example, the amount of PRP desired, the health of the donor, the severity or location of the tissue damage in the individual to be treated, the availability of prepared PRP, or any suitable combination of factors.

Any suitable amount of blood may be collected. For example, about 30 to 60 ml of whole blood may be drawn. In an exemplary embodiment, about 11 ml of blood may be withdrawn into a syringe that contains about 5 ml of an anticoagulant, such as acid-citrate-phosphate or citrate-phosphate-dextrose solution. The syringe may be attached to an apheresis needle, and primed with the anticoagulant. Blood may be drawn from the donor using standard aseptic practice. In some embodiments, a local anesthetic such as anbesol, benzocaine, lidocaine, procaine, bupivicaine, or any appropriate anesthetic known in the art may be used to anesthetize the insertion area.

The Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume. PRP may be isolated from whole blood by any method known in the art. For example, the PRP may be prepared from whole blood using a centrifuge. In a particular embodiment, whole blood is spun at 150-1350.times.g for 6 minutes at room temperature. In another embodiment, whole blood can be centrifuged using a gravitational platelet system, such as the Cell Factor Technologies GPS SYSTEM™ centrifuge. The blood-filled syringe may be slowly transferred to a disposable separation tube which may be loaded into a port on the GPS centrifuge. The sample may be capped and placed into the centrifuge. The centrifuge may be counterbalanced with a tube comprising sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood is collected from a patient, about 5 cc of PRP may be obtained. The PRP may be buffered using an alkaline buffering agent to a physiological pH. The buffering agent may be a biocompatible buffer such as HEPES, TRIS, monobasic phosphate, monobasic bicarbonate, or any suitable combination thereof capable of adjusting the PRP to physiological pH between about 6.5 and about 8.0. In certain embodiments, the physiological pH is adjusted to about pH 7.3 to about pH 7.5, more specifically, about pH 7.4. In certain embodiments, the buffering agent is an 8.4% sodium bicarbonate solution. In this embodiment, for each cc of PRP isolated from whole blood, 0.05 cc of 8.4% sodium bicarbonate may be added. In some embodiments, the syringe may be gently shaken to mix the PRP and bicarbonate. Platelet counts in the PRP can be counted and recorded, and the PRP can be resuspended for a precise number of wells in a compatible vehicle or in the donor's own plasma prior to combining with Type 2 monocytes according to the methods described herein.

In some embodiments of the compositions and methods provided herein, the composition comprises Type 2 monocytes and PRP derived from placental perfusate. An exemplary method for isolating PRP from placental perfusate is as follows. Following exsanguination of the umbilical cord and placenta, the placenta is placed in a sterile, insulated container at room temperature and delivered to the laboratory within 4 hours of birth. The placenta is discarded if, on inspection, there is evidence of physical damage such as fragmentation of the organ or avulsion of umbilical vessels. The placenta is maintained at room temperature (23.degree.+/−2.degree. C.) or refrigerated (4.degree. C.) in sterile containers for 2 to 20 hours. Periodically, the placenta is immersed and washed in sterile saline at 25.degree.+/−3.degree. C. to remove any visible surface blood or debris. The umbilical cod is transected approximately 5 cm from its insertion into the placenta and the umbilical vessels are cannulated with Teflon or polypropylene catheters connected to a sterile fluid path allowing bidirectional perfusion of the placenta and recovery of the effluent fluid.

The cannula can be, e.g., flushed with IMDM serum-free medium (GibcoBRL, NY) containing 2 U/ml heparin (Elkins-Sinn, N.J.). In one embodiment, perfusion of the placenta continues at a rate of 50 mL per minute until approximately 300-750 mL of perfusate is collected. During the course of the procedure, the placenta may be gently massaged to aid in the perfusion process and assist in the recovery of cellular material. Effluent fluid is collected from the perfusion circuit by both gravity drainage and aspiration through the arterial cannula. The perfusion and collection procedures may be repeated once or twice until the number of recovered nucleated cells falls below 100 .mu.L. The perfusates are pooled and subjected to light centrifugation to isolate platelets. Platelets can be can be resuspended for a precise number of wells in a compatible vehicle or in the donor's own plasma prior to combining with Type 2 monocytes according to the methods described herein.

In some embodiments, an individual is contacted with a combination composition comprising Type 2 monocytes and platelet rich plasma as provided herein. In a specific embodiment, said contacting is the introduction, e.g., transplantation, of said combination composition into said individual. Thus, the method of combining Type 2 monocytes with platelet rich plasma may be performed as a first step in a procedure for introducing the combination composition into any individual needing stem cells, e.g., Type 2 monocytes. Such a procedure can comprise use of pharmaceutical compositions comprising the combination compositions, as described above. Alternatively, each component of the combination composition can be introduced, e.g., transplanted into said individual serially. For example, platelet rich plasma may be administered to the individual in a first step, near the area where the pathogenesis is present, to form a stable hydrogel in vivo. In a second step, Type 2 monocytes may be administered, e.g., injected into the formed hydrogel.

In a specific embodiment, Type 2 monocytes are combined with platelet rich plasma prior to administration to an individual in need thereof in a ratio (e.g., by volume or number of cells) that results in prolonged localization of the Type 2 monocytes at the site of injection or implantation, relative to administration of Type 2 monocytes not combined with platelet rich plasma. In another specific embodiment. Type 2 monocytes are combined with platelet rich plasma during, or simultaneously with, administration to an individual in need thereof, in an optimum ratio, that results in prolonged localization of the Type 2 monocytes at a site of injection or implantation, relative to administration of Type 2 monocytes not combined with platelet rich plasma. In another specific embodiment, Type 2 monocytes and platelet rich plasma are administered sequentially to an individual in need thereof to a final optimum ratio. In one embodiment, the Type 2 monocytes are administered first and the platelet rich plasma is administered second. In another embodiment, the platelet rich plasma is administered first and the Type 2 monocytes are administered second.

In a specific embodiment, said composition comprising Type 2 monocytes and platelet rich plasma is contained within one bag or container. In another embodiment, provided herein is the use in transplantation of Type 2 monocytes, and platelet rich plasma, that are contained within separate bags or containers. In certain embodiments, Type 2 monocytes and platelet rich plasma contained in two separate bags may be mixed prior, in particular immediately prior, to or at the time of administration to an individual in need thereof.

The combining, i.e., mixing of Type 2 monocytes with platelet rich plasma to obtain the combination compositions provided herein is generally performed gently so as to not activate the platelets within the PRP.

In particular embodiments, the Type 2 monocytes and platelet rich plasma are provided in separate chambers of a 2-chamber syringe and reconstituted in the syringe prior to administration, e.g., injection into the individual.

Compositions comprising Type 2 monocytes and platelet rich plasma may be mixed, prior to transplantation, by any medically-acceptable means. In one embodiment, the two components are physically mixed. In another embodiment of the method, the Type 2 monocytes and the platelet rich plasma are mixed immediately prior to (i.e., within 1, 2, 3, 4, 5, 7, 10, 20 or 30 minutes of) administration to said individual. In another embodiment, the Type 2 monocytes and the platelet rich plasma are mixed at a point in time more than five minutes prior to administration to said individual. In another embodiment of the method, the Type 2 monocytes and/or platelet rich plasma are cryopreserved and thawed prior to administration to said individual. In another embodiment, said Type 2 monocytes and platelet rich plasma are mixed to form a composition at a point in time more than 1, 2, 3, 4, 5, 6, 7, 8, 9, 10, 11, 12, 13, 14, 15, 16, 17, 18, 19, 20, 21, 22, 23, or 24 hours prior to administration to said individual, wherein either or both of the Type 2 monocytes and platelet rich plasma have been cryopreserved and thawed prior to said administration. In another embodiment, the composition comprising Type 2 monocytes and Type 2 monocytes may be administered to an individual more than once.

In some embodiments, the platelet rich plasma component of the composition, when administered separately from the Type 2 monocytes component, can be administered as a liquid, a solid, a semi-solid (e.g., a gel), or a combination thereof. In such embodiments, when the platelet rich plasma is delivered as a liquid, it may comprise a solution, an emulsion, a suspension, or the like.

In some embodiments, a platelet rich plasma semi-solid or gel may be prepared by adding an agent to the platelet rich plasma, alone or combined with Type 2 monocytes, e.g., to better preserve the position of the Type 2 monocytes once the combination composition is delivered to the target tissue, For example, the platelet rich plasma, alone or in combination with Type 2 monocytes, may include collagen, cyanoacrylate, adhesives that cure upon injection into tissue, liquids that solidify or gel after injection into tissue, suture material, agar, gelatin, light-activated dental composite, other dental composites, silk-elastin polymers, MATRIGEL™, gelatinous protein mixture (e.g., from BD Biosciences), hydrogels and/or other suitable biopolymers. In certain other embodiments, a clotting agent (e.g., thrombin and/or calcium) may be added to the PRP above or combined with Type 2 monocytes. Alternatively, the clotting agent may be delivered to a target tissue before or after platelet rich plasma, alone or in combination with Type 2 monocytes, has been delivered to the target tissue to cause the platelet rich plasma to gel. In other embodiments, no clotting agents are added to the platelet rich plasma or to the combination composition comprising platelet rich plasma and Type 2 monocytes. In particular embodiments, the composition comprising Type 2 monocytes combined with platelet rich plasma, provided herein, does not comprise, and does not require, a clotting agent (e.g., thrombin and/or calcium) to effect prolonged localization of the Type 2 monocytes at the site of injection or implantation, relative to Type 2 monocytes not administered in combination with platelet rich plasma. For example, platelet rich plasma, alone or in combination with Type 2 monocytes, may harden or gel in response to one or more environmental or chemical factors such as temperature, pH, proteins, etc., without the addition of a clotting agent.

In another embodiment, the Type 2 monocytes contained within the composition are preconditioned prior to transplantation. In a various embodiments, preconditioning comprises storing the cells in a gas-permeable container generally for a period of time at about −5.degree. C. to about 23.degree. C., about 0.degree. C. to about 10.degree. C., or about 4.degree. C. to about 5.degree. C. The cells may be stored between 18 hours and 21 days, between 48 hours and 10 days, preferably between 3-5 days. The cells may be cryopreserved prior to preconditioning or, may be preconditioned immediately prior to administration. In some embodiments, the Type 2 monocytes may be differentiated prior to introduction of the combination composition to an individual in need of stem cells, e.g., Type 2 monocytes. The combination of differentiated Type 2 monocytes and platelet rich plasma is encompassed within the phrase “combination composition.” In certain embodiments, the method of transplantation of a combination composition provided herein comprises (a) induction of differentiation of Type 2 monocytes, (b) mixing the Type 2 monocytes with platelet rich plasma to form a combination composition, and (c) administration of the combination composition to an individual in need thereof. In certain other embodiments, the method of transplantation of a combination composition provided herein comprises (a) mixing the Type 2 monocytes with platelet rich plasma to form a combination composition, (b) induction of differentiation of Type 2 monocytes, and (c) administration of the combination composition to an individual in need thereof.

In another embodiment of the invention, type 2 monocytes are administered together with biologically useful stem cells of the mesenchymal or related lineages. The combination of these cells is administered in order to reduce suicidal attempts and/or suicidal ideations.

Stem cells useful for the invention may be therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). In some embodiments M2 macrophages are generated from pluripotent stem cells. Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of neurological diseases associated with neuroinflammation. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

The compositions provided herein, comprising Type 2 monocytes and platelet rich plasma, or each component of the composition, may be transplanted into an individual in any pharmaceutically or medically acceptable manner, including by surgical implantation or injection, e.g., intravenous injection, intraarterial injection, intra-articular injection, intramuscular injection, intraperitoneal injection, intraocular injection, direct injection into a particular tissue. The site of delivery of the composition is typically at or near the site of pathogenesis, e.g., tissue damage. The site of tissue damage can be determined by well-established methods including medical imaging, patient feedback, or a combination thereof. The particular imaging method used may be determined based on the tissue type. Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques. The patient may also assist in locating the site of tissue injury or damage by pointing out areas of particular pain and/or discomfort. The PRP composition may be delivered minimally invasively and/or surgically. For example, to deliver a PRP composition to ischemic tissue, a physician may use one of a variety of access techniques, including but not limited to, surgical (e.g., sternotomy, thoracotomy, mini-thoracotomy, sub-xiphoidal) approaches, endoscopic approaches (e.g., intercostal and transxiphoidal) and percutaneous (e.g., transvascular) approaches.

In one embodiment of the invention, umbilical cord monocytes are administered intramuscularly at a concentration of 100,000-10 billion cells.

In some embodiments of the invention, type 2 monocytes, and/or umbilical cord mononuclear cells are utilized to suppress and/or modify inflammatory and/or immunological pathways associated with suicide or factors associated with suicide. The discussion below provides means of assessing immunological characteristics associated with suicide what can be utilized to determine doseage and/or frequency of cell administration.

The problem of suicide takes a substantial toll on our society. It is the 2nd leading cause of death of victims aged 15-34 and is overall the 10th leading cause of death in the United States [1, 2]. There is a dramatic need for new approaches to suicide prevention. Despite significant efforts, the rates of suicide have been increasing for the last 20 years. Between 1999 and 2017, the suicide rate had risen by 33%, according to the U.S. Centers for Disease Control and Prevention (CDC) [3, 4]. Current efforts to prevent suicide are focused around psychological and psychiatric interventions, primarily to address underlying predisposing factors such as major depressive disorder [5, 6], bipolar disorder [7, 8], schizophrenia [9], substance abuse [10], toxoplasma infection [11], insomnia [12-15], and post-traumatic stress disorder (PTSD) [16, 17]. Unfortunately, as exemplified by the increasing rates, current interventions are primarily ineffective. An example of the sad state of affairs, a recent meta-analysis of 29 placebo-controlled studies which included 6,934 patients with major depressive disorder demonstrated no significant inhibition of suicide by antidepressants [18].

In light of the “mediocre at best” successes at addressing the problem of suicide using psychological or chemical manipulations, we propose examination of the problem of suicide from an immunological perspective. The fundamental cross-talk between the immune system and nervous system, although highly discussed throughout history, has to our knowledge never been clinically translated in the area of suicide prevention.

The potent interconnection between brain function and immune function is apparent in animals which lack immune cells such as severe combined immune deficient (SCID) mice. These animals possess significant impairment in ability to perform cognitive tasks as compared to wild-type (WT) control mice. Supporting the relevance of immunity to mental function is the fact that if one depletes WT animals of T cells, significantly impaired learning behavior results. Furthermore, it was shown that transfer of syngeneic T cells into WT mice following ablation of adaptive immunity restored previously impaired cognitive function [19].

The notion of T cells altering mental function has also been shown by studies in which mice genetically or artificially made to lack these cells are impaired in performance of cognitive tasks such as Morris water maze (MWM), Barnes maze and others. Studies have demonstrated that this is a reversible phenomenon; injection of immune deficient mice with T cells from wild type counterparts improves their cognitive function. It was further shown that macrophages alternatively activated in vitro (M2 cells) can circumvent the need for ‘pro-cognitive’ T cells when injected intravenously into immune deficient mice. Therefore T cells, as well as type 2 macrophages appear to play a role in cognitive function [20]. These studies, suggesting that immune cells can influence mental activities are further supported by studies in which immunization can reduce post stress mental anxiety [21]. Others have also reported anxiolytic [22], and memory restoring [23, 24], effects of T cells.

One of the most fundamental demonstrations of brain immune cross-talk are experiments demonstrating Pavlovian conditioning of immunity. Numerous studies in mice and man have shown feasibility of conditioning a scent or a taste with either immune suppressive [25-34], or immune stimulatory drugs [35-39]. The practical relevance of such conditioning experiments is seen in efficacy of using the conditioned stimuli alone to treat disease. For example, Bauer et al used a saccharin solution as a conditioned stimulus, which was combined with the immune suppressive drug cyclosporin. After 6 pairings of cyclosporine and saccharin, animals where induced to undergo autoimmune uveitis using a standard protocol. Decreased Th1 responses, which are suppressed by cyclosporine, but not Th17, which is not suppressed by cyclosporine, was observed [40]. This, and numerous other experiments, demonstrated that conditioning of immunity by mental association can be utilized to treat pathology [41], or induce pathology [42]. An interesting experiment used a psychological technique to “amplify” the Pavlovian conditioning effect. Luckemann et al. utilized the collagen II rheumatoid arthritis model with Pavlovian conditioning of a new taste to the rats. Saccharin, which served as the conditioned stimulus was paired with cyclosporin A. Arthritis was induced by injection of type II collagen with adjuvant. Fourteen days later, at the first occurrence of clinical symptoms, saccharin was given together with low-dose cyclosporine as reminder cues to prevent the conditioned response from being extinguished. The low-dose cyclosporine was below the concentration needed to induce a therapeutic effect in the animals. The arthritis score and histologic inflammatory symptoms were suppressed as much as was accomplished with full-dose pharmacologic treatment [43].

Numerous mechanisms of brain-immune system interactions have been elucidated. For example, it is known that all major immune organs are highly innervated. In some experiments beta adrenergic nerves have been demonstrated to be essential for Pavlovian conditioning of immune modulation [44-46]. Interestingly, other immunological events are also related to involvement of nervous system. For example, the process of bone marrow stem cell mobilization by G-CSF administration has been shown to be dependent on sympathetic nervous system [47-51], as well as endocannabinoid receptor [52], activation in the osteal bone microenvironment. Another example of the brain-immune system interplay is the prevention of sepsis associated death in animals via a vagus-nerve dependent manner, in which stimulation of the nerve is protective and inactivation is associated with hypersensitivity to inflammation induced toxicity [53-56]. The technology of vagus nerve stimulation is used to treat epilepsy in humans. Since commercially available devices exist that are clinically applicable, one study sought to determine whether stimulation of this nerve could be clinically utilized to treated rheumatoid arthritis. In a clinical study it was demonstrated that an implantable vagus nerve-stimulating device in epilepsy patients inhibits peripheral blood production of TNF, IL-1β, and IL-6. Furthermore, it was demonstrated that vagus nerve stimulation in arthritis patients significantly inhibited TNF production for up to 84 days. Moreover, RA disease severity, as measured by standardized clinical composite scores, significantly decreased [57]. Unfortunately, besides this pilot trial, there is no real evidence of clinical translation using the nervous system to modulate the immune system.

The invention provides means of decreasing neurological adverse effects of immunotherapy through administration of umbilical cord mononuclear cells and/or monocytes. Immunotherapy of cancer has been widely recognized in recent years as a breakthrough. The advent of immune checkpoint inhibitors, for which the Nobel Prize in Medicine was awarded, signaled a new era of hope for previously untreatable patients [58]. Unfortunately, immunotherapy has had a jaded past, in part because of various severe adverse events associated with its administration. One of the first types of cancer immunotherapy was administration of the cytokine interleukin-2. This protein, produced by T cells and various other cells, is considered a T cell growth factor, however it also increases activity of natural killer (NK) cells. When interleukin-2 is provided systemically, there are numerous physiological changes, including tryptophan degradation. This is believed to occur through activation of various immune cells which express the enzyme indolamine-2,3-deoxygenase (IDO). In one study, investigators assessed 16 cancer patients eligible to receive immunotherapy with interleukin-2 and/or interferon-alpha participated in the study. At baseline and after one week and one month of therapy, depressive symptoms were assessed using the Montgomery-Asberg Depression Rating Scale (MADRS), and blood samples were collected for the determination of the large neutral amino acids (LNAA) (tryptophan, tyrosine, valine, leucine, isoleucine, phenylalanine) which compete for transport across the blood-brain barrier. Serum concentrations of tryptophan as well as the tryptophan/LNAA ratio significantly decreased between baseline, one week and one month of therapy. The development and severity of depressive symptoms, especially anorexia, pessimistic thoughts, suicidal ideation and loss of concentration were positively correlated with the magnitude of the decreases in tryptophan concentrations during treatment [59]. The possibility of immune activation inducing suicidal thoughts was confirmed in other studies. The cytokine interferon beta, which possesses various immune modulatory activities has been associated with suicidal ideations. The first report of interferon associated neurological changes was a 21-year-old man with multiple sclerosis without a psychiatric history. He was administered interferon 22 microgram three times a week which resulted in recurring suicidal thoughts. Suicidal ideations resolved rapidly after interferon beta-la withdrawal [60]. In phase III trials of interferon beta, suicidal ideation was reported to be present in a significant number of patients. In numerous cases withdrawal of the medication results in regression of suicidal ideations [61-63]. Interferon alpha is another immunotherapeutic cytokine that has been shown to be effective for treatment of viral infections. Importantly, approximately 30-70% of hepatitis C virus-infected patients treated with IFN-alpha experience different degrees of depression [64]. It is reported that interferon alpha stimulates the hypothalamic pituitary adrenal axis, which is involved intra alia in activation of interleukin 6 production [65-67]. The influence of interleukin-6 in depression is well known.

The fact that immune activators administered as therapy induce depressive thoughts and suicidal ideations supports the notion that there is an immunological aspect to suicidal ideation. We will discuss factors that are associated with suicide and discuss correlations between them an immunological inflammatory activation.

In one embodiment the invention provides novel means of treating major depressive disorder (MDD). MDD is a condition associated with depression, lack of interest, anhedonia, fear, feelings of worthlessness, inability to sleep and problems maintaining concentration. It is believed that MDD has a lifetime prevalence of approximately 20%. This disease is referred to as a life-threatening illness since as many as 10% of patients with severe MDD commit suicide. Current treatments of MDD include antidepressants, electroconvulsive therapy (ECT) and ketamine.

An inflammatory basis for MDD has been proposed by several investigators. Initial findings included elevated inflammatory cytokines in the blood of patients. One of the first studies to investigate this examined data collected from 3024 well-functioning individuals of 70-79 years of age. Depressed mood was defined as a Center for Epidemiologic Studies Depression scale score of 16 or higher. Plasma concentrations of interleukin (IL)-6, tumor necrosis factor (TNF)-alpha, and C-reactive protein (CRP) were measured. Compared with the 2879 nondepressed subjects, the 145 persons with depressed mood possessed elevated plasma levels of IL-6, TNF-alpha, and CRP. After adjustment for health and demographic variables, depressed mood was especially prevalent among persons who had increase plasma level for at least two of the inflammatory markers [68]. Numerous other studies have demonstrated upregulation of plasma IL-6 in patients with MDD [69-98].

Interestingly, pointing to a pathological role of IL-6 are studies in which elevations of this acute phase protein are associated with resistance to psychiatric therapy of MDD. In one study, plasma concentration of IL-6 was assessed in ninety-eight patients with stable MDD. The investigators found a significant relationship between number of failed treatment trials and inflammatory markers including IL-6 [99]. Another study demonstrated a role of IL-6 in treatment resistance. Twenty-nine patients who suffered from MDD had levels inflammatory cytokines tested including IL-6 and severity of depressive symptoms prospectively evaluated before ECT treatment, after the second ECT session, and again at the completion of the index treatment series. The investigators reported that in multivariate analyses, higher levels of IL-6 at baseline, but not other inflammatory markers or clinical variables, were associated with lower end-of-treatment success [100].

The invention provides novel means of treatment of patients suffering from bipolar disorder (BD), previously named manic depression, is a serious mental disorder characterized by remitting and relapsing episodes of depression and mania, which can also include psychotic symptoms such as delusions and hallucinations. Manifestation of the condition usually begins late adolescence or early adulthood. The condition affects males and females equally. BD has a lifetime prevalence of 1.0% for bipolar I disorder and 1.1% for bipolar II disorder. Treatment comprises of mood stabilizers such as lithium or valproate, although their biological mechanisms are not elucidated. It is estimated that between 25% and 60% of individuals with bipolar disorder will attempt suicide at least once in their lives and between 4% and 19% will complete suicide [101].

An inflammatory basis for bipolar disorder is supported by elevated acute phase proteins and cytokines in the plasma. For example, in one study repeated measurements of plasma levels of IL-6, IL-10, IL-18, IL-1(3 and TNF-α were obtained during 6-12 months in 37 rapid cycling bipolar disorder patients and compared with repeated measurements in 40 age- and gender matched healthy control subjects. Adjusting for demographical, clinical- and lifestyle factors, levels of IL-6 and IL-18 were significantly elevated in rapid cycling bipolar disorder patients in a manic/hypomanic state, compared with a depressed and a euthymic state. Furthermore, when compared with healthy control subjects, IL-6 and IL-18 were significantly increased in manic/hypomanic bipolar disorder patients [102].

Other studies have demonstrated what appears to be correlations between inflammatory markers such as CRP [103], TNF-alpha [104, 105], IL-1 beta [106-108], and IL-6 [109, 110], with bipolar disorder. In addition to circulating cytokine differences, it appears that bipolar disorder is also associated with a propensity towards Th1 immunity. In one study, 27 female subjects with BD type I (all medicated) and 24 age- and sex-matched controls were recruited. Lymphocytes were isolated and stimulated in vitro to assess Th1/Th17/Th2 cytokines and MAPK phosphorylation. It was found that BD patients had reduced proportions of T regulatory cells as compared to healthy controls. Additionally, BD was associated with a strong bias to Th1 rather than Th2 profile. T cells of BD patients had an increased p-ERK signaling which supports the notion that lymphocytes of patients where chronically activated [111]. Numerous other studies in patients with BD have observed a predisposition towards Th1 immunity [112-117].

In one embodiment the invention disclosed a novel means of treating schizophrenia and/or augmenting efficacy of existing treatments. Schizophrenic patients have abnormalities in perception of reality and in their expression of reality. Many patients suffer from hallucinations and various types of delusions. Symptoms usually begin in young adulthood. Diagnosis of schizophrenia is performed using self-reported experiences and observed behavior. It is known that schizophrenics usually also have MDD and anxiety, as well as propensity for alcohol and substance abuse. Schizophrenics possess a significantly high propensity towards suicide.

Supporting an inflammatory basis for schizophrenia are numerous studies showing correlation between this condition and elevated cytokines, especially IL-6. In one set of experiments, Kalmady et al compared 75 schizophrenia patients who were drug naive to 102 healthy controls and observed that schizophrenics possessed higher levels of IL-6 in comparison to controls [118]. Augmentation of IL-6 in patients as compared to healthy controls, as well as reduction of IL-6 after receiving medication has been reported in clinical trials [119, 120]. In other studies, response to treatment is associated with reduction in IL-6 [121-123].

There exists data supporting correlation between IL-6 levels and extent of schizophrenia severity. For example, Frommberger et al. conducted a study in which interleukin-6 plasma levels were determined in 12 depressed and 32 schizophrenic patients during the acute severity and after remission and were compared with 12 healthy controls. Interleukin-6 plasma concentrations were elevated during the acute state either of depression or of schizophrenia when compared to controls. After remission, IL-6 concentrations in depressed and in schizophrenic patients had decreased and did not differ significantly from controls [124].

In one embodiment of the invention treatment of alcoholism and/or adjuvant treatment is disclosed. In alcoholism, Khoruts et al. examined IL-1, IL-6, and TNF-alpha in alcoholic men. TNF-alpha and IL-1 concentrations remained elevated for up to 6 month after diagnosis of alcoholism and IL-6 normalized in parallel with clinical recovery [125]. Suggestions of morphine stimulating interleukin-6 came from several animal studies. In one study, Administration of morphine (10 mg/kg) to rats increased interleukin-6 (IL-6), levels 2 to 4-fold. pretreatment of animals with the opioid receptor antagonist, naltrexone stopped the increase in IL-6. It was shown that intact ganglionic transmission was required for both effects of morphine. Elevation of IL-6 was completely abolished in adrenalectomized animals [126].

In patients suffering from opium addiction, a study examined plasma IL-4, IFN-γ, IL-6 and TGF-β The results showed that plasma levels of IL-4 and IFN-γ were significantly lower and IL-6 and TGF-β were higher in plasma taken from opium-addicted patients as compared to healthy controls [127]. In another study, scientists investigated opioid use disorder (OUD) patients undergoing methadone therapy (MMT) and followed them up for 12 weeks. Inflammatory markers were measured such as plasma tumor necrosis factor (TNF)-α, C-reactive protein (CRP), IL-6, IL-1, transforming growth factor (TGF)-β1, brain-derived neurotrophic factor (BDNF), urinary morphine tests, and plasma morphine levels at baseline and on weeks 1, 4, 8, and 12 during MMT. It was found that plasma levels of CRP, TGF-β1, and BDNF fell during MMT. Plasma IL-6 levels were significantly associated with plasma morphine levels and urinary morphine-positive (+) results. Furthermore, plasma IL-6 levels were significantly associated with poor compliance and early dropout from MMT, thus supporting the value of IL-6 as a predictor of relapse [128].

In order to assess mechanisms by which morphine induces IL-6, scientists utilized a model in which 10 mg/kg of morphine was administered to rats. A rapid and significant (2-fold) increase in plasma IL-6 was observed after morphine administration. This effect of morphine peaked within 30 min and remained elevated for at least 2 h. Central microinjection of morphine mimicked the effects of peripherally administered morphine and was completely blocked by naltrexone pretreatment. Pretreatment with a ganglionic blocker, chlorisondamine also blocked the elevation of IL-6 by morphine. This seems to implicate a role for the autonomic nervous system. In adrenalectomized animals, morphine administration did not increase IL-6 levels, whereas in adrenal demedullated animals, the effect of morphine remained intact. Thus, the adrenal cortex may be a potential source of IL-6, because IL-6 mRNA has been localized in the adrenal gland [129].

The effects of cocaine on immunological systems also seems to be associated with increased IL-6. In one study, administration of cocaine to C57/BL6 mice resulted in increased IL-6 production from splenocytes, as well as peritoneal macrophages [130]. In vitro, pericytes [131], as well as microglial cells [132], have been shown to produce IL-6 in response to cocaine. Interestingly, cocaine has also been demonstrated to increase macrophage antigen presenting cell activity [133].

To assess clinical relevance of cocaine on cytokine levels, one study compared 24 healthy controls and 12 cocaine users and observed significantly higher IL-6 levels and lower amounts of IL-10 in serum compared to controls [134]. In another study, 42 crack cocaine-dependent women and 52 healthy women were compared with respect to brain Executive Function (EF) using the Wisconsin Card Sorting Test (WCST). The group taking crack had poor performance on WSCT scores and higher plasma IL-6 levels when compared with the control group. Interestingly, IL-6 levels correlated with worsening of several WCST sub-scores [135].

Sleep apnea is a cause of numerous health abnormalities ranging from cancer, to heart failure, to organ degeneration [136]. Numerous studies have shown correlation between sleep apnea and suicide [137-145].

In one study, relationship between cytokines and excessive daytime sleepiness (EDS) was assessed. Researchers measured morning plasma levels of cytokines of 12 sleep apneics, 11 narcoleptics, 8 idiopathic hypersomniacs, and 10 normal controls. It was found that TNF alpha was significantly elevated in sleep apneics and narcoleptics compared to that in normal controls and IL-6 was markedly and significantly elevated in sleep apneics compared to that in normal controls [146].

While circulating cytokines are indicative of underlying issues, another way to examine immune proclivities is to activate immunocytes ex vivo. Liu et al examined 22 patients with obstructive sleep apnea syndrome (OSAS) and 16 normal controls. The levels of LPS-induced IL-6 and TNF-alpha expression in the supernatant of the culture of PBMC and plasma level of IL-6 and TNF-alpha in patients with OSAS were significantly higher than those in the normal controls. There were significantly positive correlation between the levels of IL-6 and TNF-alpha and the percentage of time of apnea and hyponea, as well as the percentage of time spending at SaO2 below 90% in the total sleep time [147].

In addition to numerous other studies showing elevation of IL-6 [148-157], sleep apnea has been associated with increases in various inflammatory cytokines such as interleukin-8 [158], TNF-alpha [159], interleukin-17 [160-163], interleukin-18 [164], and interleukin-23 [165, 166].

The possibility that inflammatory cytokines are involved in pathology of sleep apnea comes from a study in which the effects of etanercept, a medication that neutralizes TNF-alpha and is approved by the FDA for the treatment of rheumatoid arthritis, was observed in eight obese male apneics. These patients participated in a pilot, placebo-controlled, double-blind study during which nighttime polysomnography, multiple sleep latency test, and fasting blood glucose and plasma levels of IL-6, C-reactive protein, insulin, and adiponectin were obtained. There was a significant and marked decrease in sleepiness by etanercept, which increased sleep latency during the multiple sleep latency test by 3.1+/−1.0 min (P<0.05) compared with placebo. Also, the number of apneas/hypopneas per hour was reduced significantly by the drug compared with placebo (52.8+/−9.1 vs. 44.3+/−10.3; adjusted difference, −8.4+/−2.3; P<0.05). Furthermore, IL-6 levels were significantly decreased after etanercept administration compared with placebo (3.8+/−0.9 vs. 1.9+/−0.4 pg/ml; adjusted difference, −1.9+/−0.5; P<0.01). However, no differences were observed in etanercept vs. placebo in the levels of fasting blood glucose and plasma C-reactive protein, insulin, and adiponectin. The authors concluded that neutralizing TNFalpha activity is associated with a significant reduction of objective sleepiness in obese patients with OSA [167].

In one embodiment the invention provides a novel means of addressing post traumatic stress disorder. The condition post traumatic stress disorder (PTSD) is classified as a severe anxiety central nervous system disorder that develops in response to exposure to an event resulting in psychological trauma. PTSD is triggered by a subject witnessing or experiencing any of a wide range of events that produce intense negative feelings of fear, helplessness, or horror. This experienced fear may trigger many split-second changes in the body to prepare to defend against or avoid the danger. The “fight-or-flight” response is a healthy reaction meant to protect a person from harm. But it is believed that with PTSD, this reaction is altered. Treatments for PTSD include psychotherapy and/or medications. Sertraline (Zoloft) and paroxetine (Paxil), both of which are antidepressants, have been approved by the FDA for treating people with PTSD and are administered systemically, typically orally. Other types of systemically administered medications may also be prescribed for people suffering from PTSD, such as benzodiazepines, antipsychotics, or other antidepressants.

There are indications that correlations exist between PTSD and inflammatory markers. For example, one study performed psychometric measurement of depression (BDI-II), traumatic stress symptoms (TSC-40) and dissociation (DES, SDQ-20), and immunochemical measure of serum IL-6 in 40 patients with unipolar depression. The results showed that IL-6 is significantly correlated to BDI-II, TSC-40, SDQ-20 but not to DES. The authors concluded that increased level of IL-6 in depression could be directly related to symptoms of traumatic stress [168].

In another study, 48 patients, aged 20-60, hospitalized following various orthopedic injuries including bone fractures, and 13 healthy volunteers matched for gender. At hospitalization (Time 1), 30 ml heparinized venous blood were drawn and cytokines levels in serum were assessed; participants filled out the Acute Stress Disorder Inventory (ASDI), COPE, and injury-related questionnaires. One month later (Time 2), 26 participants filled out the Posttraumatic Disorder Symptom Scale (PDS). It was found that higher serum levels of IL-6, IL-8, and TGF-beta and lower levels of serum IL-4 and IL-10 were present on injured patients as compared with controls. When controlling for age and severity of injury in the regression analysis, higher levels of IL-6 and IL-8 and lower TGF-beta were predicted by higher (acute stress symptoms) ASS and higher use of and emotion-focused coping. Higher posttraumatic stress symptoms (PTSS) scores at Time 2 were predicted by higher levels of IL-8, lower levels of TGF-beta, and higher ASS measured at Time 1. The authors concluded that high IL-6 and IL-8 and lower TGF-beta should be further assessed as a possible risk factor or a biomarker of PTSS in accident casualties [169].

In another study, scientists included 18 men and 20 women who did not meet DSM-IV criteria for Axis I psychiatric disorders or any major medical illness. Cytokine and CRP levels were obtained from baseline blood samples. Subjects completed the Early Trauma Inventory Self Report (ETI-SR). The primary outcomes included serum interleukin-6 (IL-6), tumor necrosis factor-α (TNF-α), interleukin-1β (IL1-β), and CRP levels. In addition, the mean numbers of traumatic experiences (sexual, physical, emotional, general, and the summed total) were measured. Significant positive associations were found between the total ETI score and IL-6, IL1β, and TNF-α. Significant positive correlations were found between the number of general traumas and IL1-β, TNF-α, and IL-6 [170]. Another study examined the concentrations of TNFα, IL-6 and IL-10 and associations with behavioral symptoms, including post-traumatic stress disorder symptoms and depression in a cohort of 106 military personnel and Veterans with a history of TBI. Group comparisons conducted for those with repetitive TBIs (>3; n=44), to participants with less than three TBIs (n=29), and controls with no TBIs (n=33). The primary outcomes were serum levels of inflammatory related proteins TNF-α, IL-6 and IL-10, TBI history, and PTSD symptoms. IL-6 mean concentration was significantly higher in the repetitive TBI group compared to those with 1-2 TBI or no TBI history. Additionally, for participants with a history of TBI, PTSD symptom severity, specifically, intrusion and avoidance, were significant predictors of higher IL-6 and IL-10 concentrations respectively. These findings suggest that repetitive TBIs concurrent with high PTSD symptoms in military personnel and Veterans are associated with chronic inflammation, and specifically elevated concentrations of IL-6 [171].

In one embodiment the invention teaches a means of decreasing inflammatory cytokines. The relevance of these cytokines is discussed as follows. One of the first studies to describe relationship between suicide and inflammatory cytokines investigated levels of IL-1, IL-6, and TNF-alpha in cerebrospinal fluid of 63 suicide attempters and 47 healthy control subjects. Subjects were categorized based on diagnosis and violent or nonviolent suicide attempt. Investigators evaluated suicidal ideation and depressive symptoms using the Suicide Assessment Scale and the Montgomery-Asberg Depression Rating Scale (MADRS). It was found that IL-6 in CSF was significantly higher in suicide attempters than in healthy control subjects. Patients who performed violent suicide attempts displayed the highest IL-6. Furthermore, there was a significant positive correlation between Montgomery-Asberg Depression Rating Scale MADRS scores and CSF IL-6 levels in all patients [172].

Subsequently a study examined plasma immune related cytokines such as IL-2, IL-6 and tumor necrosis factor (TNF)-α in 47 suicide attempters, 17 non-suicidal depressed patients and 16 healthy controls. It was found that there were increased levels of IL-6 and TNF-α as well as decreased IL-2 concentrations in suicide attempters compared to non-suicidal depressed patients and healthy controls. Importantly the researchers had the results adjusted for potential confounders of cytokine expression, such as age, sex, body mass index (BMI), degree of depression, anxiety, personality disturbance, abuse and type of medication. The authors concluded that for the first time suicidal patients display a distinct peripheral blood cytokine profile compared to non-suicidal depressed patients [173]

Further supporting the notion of inflammation being associated with suicide is a study that measured inflammatory markers in patients with MDD with and without high levels of suicidal ideation and in nondepressed controls. Levels of suicidal ideation, depression severity, and recent suicide attempts were assessed by structured clinical interviews. Patients with MDD and high suicidal ideation had significantly higher inflammatory index scores (TNF, IL-6 and CRP) than both controls, and patients with MDD and lower suicidal ideation [174].

There are many potential hypotheses that could be made regarding increased inflammatory cytokine levels. In one study, researchers compared vitamin D levels in 59 suicide attempters, 17 non-suicidal depressed patients and 14 healthy controls It was found that suicide attempters had significantly lower mean levels of vitamin D than depressed non-suicidal patients and healthy controls. 58 percent of the suicide attempters were vitamin D deficient according to clinical standard. Furthermore, researchers found that there was a significant negative association between vitamin D and pro-inflammatory cytokines in the psychiatric patients [175]

Supporting causality of IL-6, in another study, 92 patients with major depressive disorder and/or anxiety disorders, were treated with fluoxetine (FLX) for 8 weeks. Plasma concentrations of TNFα, IL-6, and IL-1β were measured by enzyme linked immunosorbent assays before and after FLX treatment. The investigators found that IL-6 levels increased after treatment only in the group of children who developed FLX-associated suicidality [176].

In a significantly large validation study, 2329 adults with current or remitted depression and/or anxiety enrolled in the Netherlands Study of Depression and Anxiety. Sleep duration, insomnia, past week suicidal ideation (SI), and suicide attempts (SA) were assessed with self-report measures. It was found that short sleep duration (≤6 h) compared to normal sleep duration (7-9 h) was associated SA. A higher likelihood of SI during the past week was observed for participants with long sleep duration (≥10 h) compared to normal sleep duration, more insomnia symptoms, and higher IL-6 [177].

### EXAMPLES

### Suppression of Inflammation Induced Memory Dysfunction by Cord Blood Derived Monocytes

The lipopolysaccharide administration model was used to replicate neuroinflammation associated with suicidal behavior. Female BALB/c mice were treated with control, daily lipopolysaccharide treatment (10 ng/mouse0, and some with cord blood derived monocytes. Cord blood derived monocytes were extracted by ficoll separation of cord blood mononuclear cells and 4-hour incubation on T175 plastic flasks. Non-adherent cells were washed off by rinsing with phosphate buffered saline and adherent cells where subsequently dissociated using trypsin and placed into another flask. Cord blood monocytes where cultured for 24 hours with 1 IU of oxytocin per million cells in complete DMEM media with 10% fetal calf serum. Cells were dissociated with trypsin, washed in phosphate buffered saline and administered intravenously vial tail vein at a concentration of 500,000 per mouse on days 0, 3, and 6.

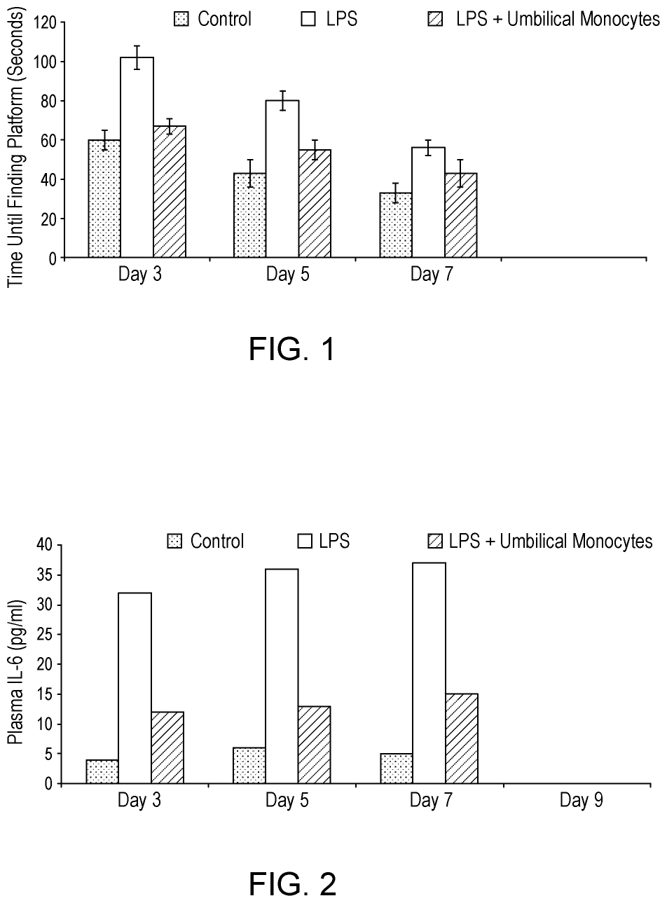

To assess memory function, water filled basin which was 120 cm in diameter was broken into 4 quadrants. 10 cm diameter platform placed 1 cm below water. Mice were forced to swim to find the hidden platform, starting from all four different quadrants, each day for a total of 7 days. As seen below, mice receiving umbilical cord monocytes resisted the pathological effects of lipopolysaccharide treatment. Results are shown in FIG. 1.

### Suppression of LPS Induced Plasma Interleukin-6 by Cord Blood Derived Monocytes

The lipopolysaccharide administration model was used to replicate neuroinflammation associated with suicidal behavior. Female BALB/c mice were treated with control, daily lipopolysaccharide treatment (10 ng/mouse0, and some with cord blood derived monocytes. Cord blood derived monocytes were extracted by ficoll separation of cord blood mononuclear cells and 4-hour incubation on T175 plastic flasks. Non-adherent cells were washed off by rinsing with phosphate buffered saline and adherent cells where subsequently dissociated using trypsin and placed into another flask. Cord blood monocytes where cultured for 24 hours with 1 IU of oxytocin per million cells in complete DMEM media with 10% fetal calf serum. Cells were dissociated with trypsin, washed in phosphate buffered saline and administered intravenously vial tail vein at a concentration of 500,000 per mouse on days 0, 3, and 6.

To assess interleukin 6 levels in plasma, mice were sacrificed at the indicated timepoints and cytokine was assayed by ELISA. Results are shown in FIG. 2

### REFERENCES

1. Statistics:, N.C.f.H., in In: Health, United States, 2011: With Special Feature on Socioeconomic Status and Health. EDN Hyattsville, Md:. 2012.

2. Cross, W. F., et al., A randomized controlled trial of suicide prevention training for primary care providers: a study protocol. BMC Med Educ, 2019. 19(1): p. 58.

3. Lennon, J. C., Unintentional injury fatalities in the context of rising U.S. suicide rates: A five-year review of the web-based injury statistics query and reporting system. Psychiatry Res, 2020. 289: p. 113066.

4. Dec. 6 2020]; Available from: https://www.apa.org/monitor/2019/07-08/cover-prevent-suicide.

5. Moller, Moller, C. I., et al., Relationships Between Different Dimensions of Social Support and Suicidal Ideation in Young People with Major Depressive Disorder. J Affect Disord, 2020.

6. Dong, M., et al., Prevalence of suicide attempt in individuals with major depressive disorder: a meta-analysis of observational surveys. Psychol Med, 2019. 49(10): p. 1691-1704.

7. Miller, J. N. and D. W. Black, Bipolar Disorder and Suicide: a Review. Curr Psychiatry Rep, 2020. 22(2): p. 6.

8. Beyer, J. L. and R. H. Weisler, Suicide Behaviors in Bipolar Disorder: A Review and Update for the Clinician. Psychiatr Clin North Am, 2016. 39(1): p. 111-23.

9. Lu, L., et al., Prevalence of suicide attempts in individuals with schizophrenia: a meta-analysis of observational studies. Epidemiol Psychiatr Sci, 2019. 29: p. e39.

10. Yuan, S., H. Yao, and S. C. Larsson, Associations of cigarette smoking with psychiatric disorders: evidence from a two-sample Mendelian randomization study. Sci Rep, 2020. 10(1): p. 13807.

11. Sutterland, A. L., et al., Driving us mad: the association of Toxoplasma gondii with suicide attempts and traffic accidents—a systematic review and meta-analysis. Psychol Med, 2019. 49(10): p. 1608-1623.

12. Harris, L. M., et al., Sleep disturbances as risk factors for suicidal thoughts and behaviours: a meta-analysis of longitudinal studies. Sci Rep, 2020. 10(1): p. 13888.

13. Liu, R. T., et al., Sleep and suicide: A systematic review and meta-analysis of longitudinal studies. Clin Psychol Rev, 2020. 81: p. 101895.

14. Wang, X., S. Cheng, and H. Xu, Systematic review and meta-analysis of the relationship between sleep disorders and suicidal behaviour in patients with depression. BMC Psychiatry, 2019. 19(1): p. 303.

15. Liu, J. W., et al., Associations between sleep disturbances and suicidal ideation, plans, and attempts in adolescents: a systematic review and meta-analysis. Sleep, 2019. 42(6).

16. Ng, Q. X., et al., Early life sexual abuse is associated with increased suicide attempts: An update meta-analysis. J Psychiatr Res, 2018. 99: p. 129-141.

17. Siddaway, A. P., et al., A meta-analysis of perceptions of defeat and entrapment in depression, anxiety problems, posttraumatic stress disorder, and suicidality. J Affect Disord, 2015. 184: p. 149-59.

18. Braun, C., et al., Suicides and Suicide Attempts during Long-Term Treatment with Antidepressants: A Meta-Analysis of 29 Placebo-Controlled Studies Including 6,934 Patients with Major Depressive Disorder. Psychother Psychosom, 2016. 85(3): p. 171-9.

19. Brynskikh, A., et al., Adaptive immunity affects learning behavior in mice. Brain Behav Immun, 2008. 22(6): p. 861-9.

20. Derecki, N. C., K. M. Quinnies, and J. Kipnis, Alternatively activated myeloid (M2) cells enhance cognitive function in immune compromised mice. Brain Behav Immun, 2011. 25(3): p. 379-85.

21. Lewitus, G. M., H. Cohen, and M. Schwartz, Reducing post-traumatic anxiety by immunization. Brain Behav Immun, 2008. 22(7): p. 1108-1114.

22. Clark, S. M., et al., CD4(+) T cells confer anxiolytic and antidepressant-like effects, but enhance fear memory processes in Rag2(−/−) mice. Stress, 2016. 19(3): p. 303-11.

23. Clark, S. M., et al., Neonatal adoptive transfer of lymphocytes rescues social behaviour during adolescence in immune-deficient mice. Eur J Neurosci, 2018. 47(8): p. 968-978.

24. Radjavi, A., I. Smirnov, and J. Kipnis, Brain antigen-reactive CD4+ T cells are sufficient to support learning behavior in mice with limited T cell repertoire. Brain Behav Immun, 2014. 35: p. 58-63.

25. Horbelt, T., et al., Behaviorally conditioned immunosuppression with cyclosporine A forms long lasting memory trace. Behav Brain Res, 2019. 376: p. 112208.

26. Luckemann, L., et al., Behavioral conditioning of anti-proliferative and immunosuppressive properties of the mTOR inhibitor rapamycin. Brain Behav Immun, 2019. 79: p. 326-331.

27. Kirchhof, J., et al., Learned immunosuppressive placebo responses in renal transplant patients. Proc Natl Acad Sci U S A, 2018. 115(16): p. 4223-4227.

28. Wirth, T., et al., Repeated recall of learned immunosuppression: evidence from rats and men. Brain Behav Immun, 2011. 25(7): p. 1444-51.

29. Goebel, M. U., et al., Behavioral conditioning of immunosuppression is possible in humans. FASEB J, 2002. 16(14): p. 1869-73.

30. Roudebush, R. E. and H. U. Bryant, Conditioned immunosuppression of a murine delayed type hypersensitivity response: dissociation from corticosterone elevation. Brain Behav Immun, 1991. 5(3): p. 308-17.

31. MacQueen, G. M. and S. Siegel, Conditional immunomodulation following training with cyclophosphamide. Behav Neurosci, 1989. 103(3): p. 638-47.

32. Kusnecov, A. W., A. J. Husband, and M. G. King, Behaviorally conditioned suppression of mitogen-induced proliferation and immunoglobulin production: effect of time span between conditioning and reexposure to the conditioning stimulus. Brain Behav Immun, 1988. 2(3): p. 198-211.

33. Lysle, D. T., et al., Pavlovian conditioning of shock-induced suppression of lymphocyte reactivity: acquisition, extinction, and preexposure effects. Life Sci, 1988. 42(22): p. 2185-94.

34. Gorczynski, R. M., Analysis of lymphocytes in, and host environment of mice showing conditioned immunosuppression to cyclophosphamide. Brain Behav Immun, 1987. 1(1): p. 21-35.

35. Janz, L. J., et al., Pavlovian conditioning of LPS-induced responses: effects on corticosterone, splenic NE, and IL-2 production. Physiol Behav, 1996. 59(6): p. 1103-9.

36. Gorczynski, R. M., Toward an understanding of the mechanisms of classical conditioning of antibody responses. J Gerontol, 1991. 46(4): p. P152-6.

37. Kusnecov, A., M. G. King, and A. J. Husband, Immunomodulation by behavioural conditioning. Biol Psychol, 1989. 28(1): p. 25-39.

38. Hiramoto, R. N., et al., Regulation of natural immunity (NK activity) by conditioning. Ann N Y Acad Sci, 1987. 496: p. 545-52.

39. Neveu, P. J., R. Dantzer, and M. Le Moal, Behaviorally conditioned suppression of mitogen-induced lymphoproliferation and antibody production in mice. Neurosci Lett, 1986. 65(3): p. 293-8.

40. Bauer, D., et al., Behavioral Conditioning of Immune Responses with Cyclosporine A in a Murine Model of Experimental Autoimmune Uveitis. Neuroimmunomodulation, 2017. 24(2): p. 87-99.

41. Lysle, D. T., L. J. Luecken, and K. A. Maslonek, Suppression of the development of adjuvant arthritis by a conditioned aversive stimulus. Brain Behav Immun, 1992. 6(1): p. 64-73.

42. Blom, J. M., et al., Learned immunosuppression is associated with an increased risk of chemically-induced tumors. Neuroimmunomodulation, 1995. 2(2): p. 92-9.

43. Luckemann, L., et al., Learned Immunosuppressive Placebo Response Attenuates Disease Progression in a Rodent Model of Rheumatoid Arthritis. Arthritis Rheumatol, 2020. 72(4): p. 588-597.

44. Pacheco-Lopez, G., et al., Calcineurin inhibition in splenocytes induced by pavlovian conditioning. FASEB J, 2009. 23(4): p. 1161-7.

45. Hsueh, C. M., et al., Involvement of catecholamines in recall of the conditioned NK cell response. J Neuroimmunol, 1999. 94(1-2): p. 172-81.

46. Coussons-Read, M. E., L. A. Dykstra, and D. T. Lysle, Pavlovian conditioning of morphine-induced alterations of immune status: evidence for peripheral beta-adrenergic receptor involvement. Brain Behav Immun, 1994. 8(3): p. 204-17.

47. Saba, F., et al., G-CSF induces up-regulation of CXCR4 expression in human hematopoietic stem cells by beta-adrenergic agonist. Hematology, 2015. 20(8): p. 462-468.

48. Pasupuleti, L. V., et al., Do all beta-blockers attenuate the excess hematopoietic progenitor cell mobilization from the bone marrow following trauma/hemorrhagic shock? J Trauma Acute Care Surg, 2014. 76(4): p. 970-5.

49. Chen, C., et al., Adrenaline administration promotes the efficiency of granulocyte colony stimulating factor-mediated hematopoietic stem and progenitor cell mobilization in mice. Int J Hematol, 2013. 97(1): p. 50-7.

50. Spiegel, A., et al., Catecholaminergic neurotransmitters regulate migration and repopulation of immature human CD34+ cells through Wnt signaling. Nat Immunol, 2007. 8(10): p. 1123-31.

51. Katayama, Y., et al., Signals from the sympathetic nervous system regulate hematopoietic stem cell egress from bone marrow. Cell, 2006. 124(2): p. 407-21.

52. Kose, S., et al., Human bone marrow mesenchymal stem cells secrete endocannabinoids that stimulate in vitro hematopoietic stem cell migration effectively comparable to beta-adrenergic stimulation. Exp Hematol, 2018. 57: p. 30-41 e1.

53. Wang, H., et al., Nicotinic acetylcholine receptor alpha7 subunit is an essential regulator of inflammation. Nature, 2003. 421(6921): p. 384-8.

54. Tracey, K. J., The inflammatory reflex. Nature, 2002. 420(6917): p. 853-9.

55. Borovikova, L. V., et al., Role of vagus nerve signaling in CNI-1493-mediated suppression of acute inflammation. Auton Neurosci, 2000. 85(1-3): p. 141-7.

56. Borovikova, L. V., et al., Vagus nerve stimulation attenuates the systemic inflammatory response to endotoxin. Nature, 2000. 405(6785): p. 458-62.

57. Koopman, F. A., et al., Vagus nerve stimulation inhibits cytokine production and attenuates disease severity in rheumatoid arthritis. Proc Natl Acad Sci U S A, 2016. 113(29): p. 8284-9.

58. Hafner, S. J., High hopes and high honours for cancer immunotherapy. Biomed J, 2019. 42(5): p. 293-298.

59. Capuron, L., et al., Association between decreased serum tryptophan concentrations and depressive symptoms in cancer patients undergoing cytokine therapy. Mol Psychiatry, 2002. 7(5): p. 468-73.

60. Lana-Peixoto, M. A., A. L. Teixeira, Jr., and V. G. Haase, Interferon beta-1a-induced depression and suicidal ideation in multiple sclerosis. Arq Neuropsiquiatr, 2002. 60(3-B): p. 721-4.

61. Giovannoni, G., et al., Immunogenicity and tolerability of an investigational formulation of interferon-beta1a: 24- and 48-week interim analyses of a 2-year, single-arm, historically controlled, phase IIIb study in adults with multiple sclerosis. Clin Ther, 2007. 29(6): p. 1128-45.

62. Goeb, J. L., et al., Psychiatric side effects of interferon-beta in multiple sclerosis. Eur Psychiatry, 2006. 21(3): p. 186-93.

63. Ziemssen, T., Multiple sclerosis beyond EDSS: depression and fatigue. J Neurol Sci, 2009. 277 Suppl 1: p. S37-41.

64. Schaefer, M., et al., Hepatitis C infection, antiviral treatment and mental health: a European expert consensus statement. J Hepatol, 2012. 57(6): p. 1379-90.

65. Cassidy, E. M., et al., Acute effects of low-dose interferon-alpha on serum cortisol and plasma interleukin-6. J Psychopharmacol, 2002. 16(3): p. 230-4.

66. Capuron, L., et al., Association of exaggerated HPA axis response to the initial injection of interferon-alpha with development of depression during interferon-alpha therapy. Am J Psychiatry, 2003. 160(7): p. 1342-5.

67. Udina, M., et al., Serotonin and interleukin-6: the role of genetic polymorphisms in IFN-induced neuropsychiatric symptoms. Psychoneuroendocrinology, 2013. 38(9): p. 1803-13.

68. Penninx, B. W., et al., Inflammatory markers and depressed mood in older persons: results from the Health, Aging and Body Composition study. Biol Psychiatry, 2003. 54(5): p. 566-72.

69. Yoshimura, R., T. Kishi, and N. Iwata, Plasma levels of IL-6 in patients with untreated major depressive disorder: comparison with catecholamine metabolites. Neuropsychiatr Dis Treat, 2019. 15: p. 2655-2661.

70. Xia, Q. R., et al., Increased plasma nesfatin-1 levels may be associated with corticosterone, IL-6, and CRP levels in patients with major depressive disorder. Clin Chim Acta, 2018. 480: p. 107-111.

71. Maes, M., et al., Relationships between interleukin-6 activity, acute phase proteins, and function of the hypothalamic-pituitary-adrenal axis in severe depression. Psychiatry Res, 1993. 49(1): p. 11-27.

72. Maes, M., et al., Relationships between lower plasma L-tryptophan levels and immune-inflammatory variables in depression. Psychiatry Res, 1993. 49(2): p. 151-65.

73. Sluzewska, A., et al., Indicators of immune activation in major depression. Psychiatry Res, 1996. 64(3): p. 161-7.

74. Brambilla, F. and M. Maggioni, Blood levels of cytokines in elderly patients with major depressive disorder. Acta Psychiatr Scand, 1998. 97(4): p. 309-13.

75. Musselman, D. L., et al., Higher than normal plasma interleukin-6 concentrations in cancer patients with depression: preliminary findings. Am J Psychiatry, 2001. 158(8): p. 1252-7.

76. Rief, W., et al., Immunological differences between patients with major depression and somatization syndrome. Psychiatry Res, 2001. 105(3): p. 165-74.

77. Ushiroyama, T., A. Ikeda, and M. Ueki, Elevated plasma interleukin-6 (IL-6) and soluble IL-6 receptor concentrations in menopausal women with and without depression. Int J Gynaecol Obstet, 2002. 79(1): p. 51-2.

78. Glaser, R., et al., Mild depressive symptoms are associated with amplified and prolonged inflammatory responses after influenza virus vaccination in older adults. Arch Gen Psychiatry, 2003. 60(10): p. 1009-14.

79. Lesperance, F., et al., The association between major depression and levels of soluble intercellular adhesion molecule 1, interleukin-6, and C-reactive protein in patients with recent acute coronary syndromes. Am J Psychiatry, 2004. 161(2): p. 271-7.

80. Alesci, S., et al., Major depression is associated with significant diurnal elevations in plasma interleukin-6 levels, a shift of its circadian rhythm, and loss of physiological complexity in its secretion: clinical implications. J Clin Endocrinol Metab, 2005. 90(5): p. 2522-30.

81. Nagata, T., et al., Relationship between plasma concentrations of cytokines, ratio of CD4 and CD8, lymphocyte proliferative responses, and depressive and anxiety state in bulimia nervosa. J Psychosom Res, 2006. 60(1): p. 99-103.

82. Humphreys, D., et al., Interleukin-6 production and deregulation of the hypothalamic-pituitary-adrenal axis in patients with major depressive disorders. Endocrine, 2006. 30(3): p. 371-6.

83. Bremmer, M. A., et al., Inflammatory markers in late-life depression: results from a population-based study. J Affect Disord, 2008. 106(3): p. 249-55.

84. Jacobson, C. M., et al., Depression and IL-6 blood plasma concentrations in advanced cancer patients. Psychosomatics, 2008. 49(1): p. 64-6.

85. Dimopoulos, N., et al., Increased plasma levels of 8-iso-PGF2alpha and IL-6 in an elderly population with depression. Psychiatry Res, 2008. 161(1): p. 59-66.

86. Gabbay, V., et al., Immune system dysregulation in adolescent major depressive disorder. J Affect Disord, 2009. 115(1-2): p. 177-82.

87. Jehn, C. F., et al., Association of IL-6, hypothalamus-pituitary-adrenal axis function, and depression in patients with cancer. Integr Cancer Ther, 2010. 9(3): p. 270-5.

88. Grassi-Oliveira, R., et al., Interleukin-6 and verbal memory in recurrent major depressive disorder. Neuro Endocrinol Lett, 2011. 32(4): p. 540-4.

89. Henje Blom, E., et al., Pro-inflammatory cytokines are elevated in adolescent females with emotional disorders not treated with SSRIs. J Affect Disord, 2012. 136(3): p. 716-23.

90. Hiles, S. A., et al., A meta-analysis of differences in IL-6 and IL-10 between people with and without depression: exploring the causes of heterogeneity. Brain Behav Immun, 2012. 26(7): p. 1180-8.

91. Patas, K., et al., Association between serum brain-derived neurotrophic factor and plasma interleukin-6 in major depressive disorder with melancholic features. Brain Behav Immun, 2014. 36: p. 71-9.

92. Oglodek, E. A., et al., Comparison of chemokines (CCL-5 and SDF-1), chemokine receptors (CCR-5 and CXCR-4) and IL-6 levels in patients with different severities of depression. Pharmacol Rep, 2014. 66(5): p. 920-6.

93. Vogelzangs, N., et al., Cytokine production capacity in depression and anxiety. Transl Psychiatry, 2016. 6(5): p. e825.

94. Cassano, P., et al., Inflammatory cytokines in major depressive disorder: A case-control study. Aust N Z J Psychiatry, 2017. 51(1): p. 23-31.

95. Zheng, T., et al., Increased Dipeptidyl Peptidase-4 Activity Is Associated With High Prevalence of Depression in Middle-Aged and Older Adults: A Cross-Sectional Study. J Clin Psychiatry, 2016. 77(10): p. e1248-e1255.

96. Chen, Y., et al., The Role of Cytokines in the Peripheral Blood of Major Depressive Patients. Clin Lab, 2017. 63(7): p. 1207-1212.

97. Wang, M., et al., The level of IL-6 was associated with sleep disturbances in patients with major depressive disorder. Neuropsychiatr Dis Treat, 2019. 15: p. 1695-1700.

98. Jin, K., et al., Linking peripheral IL-6, IL-1 beta and hypocretin-1 with cognitive impairment from major depression. J Affect Disord, 2020. 277: p. 204-211.

99. Haroon, E., et al., Antidepressant treatment resistance is associated with increased inflammatory markers in patients with major depressive disorder. Psychoneuroendocrinology, 2018. 95: p. 43-49.

100. Kruse, J. L., et al., Inflammation and Improvement of Depression Following Electroconvulsive Therapy in Treatment-Resistant Depression. J Clin Psychiatry, 2018. 79(2).

101. Novick, D. M., H. A. Swartz, and E. Frank, Suicide attempts in bipolar I and bipolar II disorder: a review and meta-analysis of the evidence. Bipolar Disord, 2010. 12(1): p. 1-9.

102. Munkholm, K., et al., Elevated levels of IL-6 and IL-18 in manic and hypomanic states in rapid cycling bipolar disorder patients. Brain Behav Immun, 2015. 43: p. 205-13.

103. Edberg, D., et al., Plasma C-reactive protein levels in bipolar depression during cyclooxygenase-2 inhibitor combination treatment. J Psychiatr Res, 2018. 102: p. 1-7.

104. Lee, S. Y., et al., ALDH2 modulated changes in cytokine levels and cognitive function in bipolar disorder: A 12-week follow-up study. Aust N Z J Psychiatry, 2018. 52(7): p. 680-689.

105. Kageyama, Y., et al., The relationship between circulating mitochondrial DNA and inflammatory cytokines in patients with major depression. J Affect Disord, 2018. 233: p. 15-20.

106. Uint, L., et al., Increased levels of plasma IL-1b and BDNF can predict resistant depression patients. Rev Assoc Med Bras (1992), 2019. 65(3): p. 361-369.

107. Lesh, T. A., et al., Cytokine alterations in first-episode schizophrenia and bipolar disorder: relationships to brain structure and symptoms. J Neuroinflammation, 2018. 15(1): p. 165.

108. Sanjay, T. N., et al., Plasma interleukin-6 in remitted early bipolar I disorder and subjects at high-risk for bipolar disorder. Asian J Psychiatr, 2017. 30: p. 212-213.

109. Sundaresh, A., et al., IL6/IL6R genetic diversity and plasma IL6 levels in bipolar disorder: An Indo-French study. Heliyon, 2019. 5(1): p. e01124.

110. Munkholm, K., et al., A multisystem composite biomarker as a preliminary diagnostic test in bipolar disorder. Acta Psychiatr Scand, 2019. 139(3): p. 227-236.

111. do Prado, C. H., et al., Reduced regulatory T cells are associated with higher levels of Th1/TH17 cytokines and activated MAPK in type 1 bipolar disorder. Psychoneuroendocrinology, 2013. 38(5): p. 667-76.

112. Maes, M. and A. F. Carvalho, The Compensatory Immune-Regulatory Reflex System (CIRS) in Depression and Bipolar Disorder. Mol Neurobiol, 2018. 55(12): p. 8885-8903.

113. Becking, K., et al., The circulating levels of CD4+ t helper cells are higher in bipolar disorder as compared to major depressive disorder. J Neuroimmunol, 2018. 319: p. 28-36.

114. Brambilla, P., et al., Increased M1/decreased M2 signature and signs of Th1/Th2 shift in chronic patients with bipolar disorder, but not in those with schizophrenia. Transl Psychiatry, 2014. 4: p. e406.

115. Drexhage, R. C., et al., The mononuclear phagocyte system and its cytokine inflammatory networks in schizophrenia and bipolar disorder. Expert Rev Neurother, 2010. 10(1): p. 59-76.

116. Barbosa, I. G., et al., Monocyte and lymphocyte activation in bipolar disorder: a new piece in the puzzle of immune dysfunction in mood disorders. Int J Neuropsychopharmacol, 2014. 18(1).

117. Drexhage, R. C., et al., The activation of monocyte and T cell networks in patients with bipolar disorder. Brain Behav Immun, 2011. 25(6): p. 1206-13.

118. Kalmady, S. V., et al., Plasma cytokines in minimally treated schizophrenia. Schizophr Res, 2018. 199: p. 292-296.

119. Maes, M., et al., Interleukin-2 and interleukin-6 in schizophrenia and mania: effects of neuroleptics and mood stabilizers. J Psychiatr Res, 1995. 29(2): p. 141-52.

120. Maes, M., H. Y. Meltzer, and E. Bosmans, Immune-inflammatory markers in schizophrenia: comparison to normal controls and effects of clozapine. Acta Psychiatr Scand, 1994. 89(5): p. 346-51.

121. Xu, H. M., J. Wei, and G. P. Hemmings, Changes of plasma concentrations of interleukin-1 alpha and interleukin-6 with neuroleptic treatment for schizophrenia. Br J Psychiatry, 1994. 164(2): p. 251-3.

122. Pollmacher, T., D. Hinze-Selch, and J. Mullington, Effects of clozapine on plasma cytokine and soluble cytokine receptor levels. J Clin Psychopharmacol, 1996. 16(5): p. 403-9.

123. Kim, Y. K., et al., Cytokine changes and tryptophan metabolites in medication-naive and medication -free schizophrenic patients. Neuropsychobiology, 2009. 59(2): p. 123-9.

124. Frommberger, U. H., et al., Interleukin-6-(IL-6) plasma levels in depression and schizophrenia: comparison between the acute state and after remission. Eur Arch Psychiatry Clin Neurosci, 1997. 247(4): p. 228-33.

125. Khoruts, A., et al., Circulating tumor necrosis factor, interleukin-1 and interleukin-6 concentrations in chronic alcoholic patients. Hepatology, 1991. 13(2): p. 267-76.

126. Houghtling, R. A., et al., Acute effects of morphine on blood lymphocyte proliferation and plasma IL-6 levels. Ann N Y Acad Sci, 2000. 917: p. 771-7.

127. Nabati, S., et al., The plasma levels of the cytokines in opium-addicts and the effects of opium on the cytokines secretion by their lymphocytes. Immunol Lett, 2013. 152(1): p. 42-6.

128. Lu, R. B., et al., Correlation between interleukin-6 levels and methadone maintenance therapy outcomes. Drug Alcohol Depend, 2019. 204: p. 107516.

129. Houghtling, R. A. and B. M. Bayer, Rapid elevation of plasma interleukin-6 by morphine is dependent on autonomic stimulation of adrenal gland. J Pharmacol Exp Ther, 2002. 300(1): p. 213-9.

130. Wang, Y., D. S. Huang, and R. R. Watson, In vivo and in vitro cocaine modulation on production of cytokines in C57BL/6 mice. Life Sci, 1994. 54(6): p. 401-11.

131. Sil, S., et al., Cocaine Mediated Neuroinflammation: Role of Dysregulated Autophagy in Pericytes. Mol Neurobiol, 2019. 56(5): p. 3576-3590.

132. Liao, K., et al., Cocaine-mediated induction of microglial activation involves the ER stress-TLR2 axis. J Neuroinflammation, 2016. 13: p. 33.

133. Shen, H. M., et al., Stimulation of mouse macrophage antigen presentation by cocaine. Immunopharmacol Immunotoxicol, 1999. 21(4): p. 739-53.

134. Moreira, F. P., et al., Cocaine abuse and effects in the serum levels of cytokines IL-6 and IL-10. Drug Alcohol Depend, 2016. 158: p. 181-5.

135. Levandowski, M. L., et al., Plasma interleukin-6 and executive function in crack cocaine-dependent women. Neurosci Lett, 2016. 628: p. 85-90.

136. Farrell, P. C. and G. Richards, Recognition and treatment of sleep-disordered breathing: an important component of chronic disease management. J Transl Med, 2017. 15(1): p. 114.

137. Fernandes, S. N., et al., When Night Falls Fast: Sleep and Suicidal Behavior Among Adolescents and Young Adults. Child Adolesc Psychiatr Clin N Am, 2021. 30(1): p. 269-282.

138. Yang, Y., et al., Risk of suicidal ideation, suicide attempts, and suicide deaths in persons with sleep apnea: Protocol for a systematic review and meta-analysis. PLoS One, 2020. 15(7): p. e0235379.

139. Tseng, W. C., et al., Sleep apnea may be associated with suicidal ideation in adolescents. Eur Child Adolesc Psychiatry, 2019. 28(5): p. 635-643.

140. Timkova, V., et al., Suicidal ideation in patients with obstructive sleep apnoea and its relationship with disease severity, sleep-related problems and social support. J Health Psychol, 2020. 25(10-11): p. 1450-1461.

141. Bishop, T. M., L. Ashrafioun, and W. R. Pigeon, The Association Between Sleep Apnea and Suicidal Thought and Behavior: An Analysis of National Survey Data. J Clin Psychiatry, 2018. 79(1).

142. Choi, S. J., et al., Suicidal ideation and insomnia symptoms in subjects with obstructive sleep apnea syndrome. Sleep Med, 2015. 16(9): p. 1146-50.

143. Krahn, L. E., B. W. Miller, and L. R. Bergstrom, Rapid resolution of intense suicidal ideation after treatment of severe obstructive sleep apnea. J Clin Sleep Med, 2008. 4(1): p. 64-5.

144. Bernert, R. A., et al., Suicidality and sleep disturbances. Sleep, 2005. 28(9): p. 1135-41.

145. Agargun, M. Y. and L. Besiroglu, Sleep and suicidality: do sleep disturbances predict suicide risk? Sleep, 2005. 28(9): p. 1039-40.

146. Vgontzas, A. N., et al., Elevation of plasma cytokines in disorders of excessive daytime sleepiness: role of sleep disturbance and obesity. J Clin Endocrinol Metab, 1997. 82(5): p. 1313-6.

147. Liu, H., et al., The change of interleukin-6 and tumor necrosis factor in patients with obstructive sleep apnea syndrome. J Tongji Med Univ, 2000. 20(3): p. 200-2.

148. Imagawa, S., et al., Interleukin-6 and tumor necrosis factor-alpha in patients with obstructive sleep apnea-hypopnea syndrome. Respiration, 2004. 71(1): p. 24-9.

149. Teramoto, S., H. Yamamoto, and Y. Ouchi, Increased plasma interleukin-6 is associated with the pathogenesis of obstructive sleep apnea syndrome. Chest, 2004. 125(5): p. 1964-5; author reply 1965.

150. von Kanel, R., et al., Poor sleep is associated with higher plasma proinflammatory cytokine interleukin-6 and procoagulant marker fibrin D-dimer in older caregivers of people with Alzheimer's disease. J Am Geriatr Soc, 2006. 54(3): p. 431-7.

151. Tauman, R., L. M. O'Brien, and D. Gozal, Hypoxemia and obesity modulate plasma C-reactive protein and interleukin-6 levels in sleep-disordered breathing. Sleep Breath, 2007. 11(2): p. 77-84.

152. Gozal, D., et al., Systemic inflammation in non-obese children with obstructive sleep apnea. Sleep Med, 2008. 9(3): p. 254-9.

153. Kurt, O. K., M. Tosun, and F. Talay, Serum cardiotrophin-1 and IL-6 levels in patients with obstructive sleep apnea syndrome. Inflammation, 2013. 36(6): p. 1344-7.

154. Gileles-Hillel, A., et al., Inflammatory markers and obstructive sleep apnea in obese children: the NANOS study. Mediators Inflamm, 2014. 2014: p. 605280.

155. Gaines, J., et al., Inflammation mediates the association between visceral adiposity and obstructive sleep apnea in adolescents. Am J Physiol Endocrinol Metab, 2016. 311(5): p. E851-E858.

156. Motamedi, V., et al., Elevated tau and interleukin-6 concentrations in adults with obstructive sleep apnea. Sleep Med, 2018. 43: p. 71-76.

157. Campos-Rodriguez, F., et al., Interleukin 6 as a marker of depression in women with sleep apnea. J Sleep Res, 2020: p. e13035.

158. Ke, D., et al., Enhanced interleukin-8 production in mononuclear cells in severe pediatric obstructive sleep apnea. Allergy Asthma Clin Immunol, 2019. 15: p. 23.

159. Alberti, A., et al., Plasma cytokine levels in patients with obstructive sleep apnea syndrome: a preliminary study. J Sleep Res, 2003. 12(4): p. 305-11.

160. Ye, J., et al., The treg/th17 imbalance in patients with obstructive sleep apnoea syndrome. Mediators Inflamm, 2012. 2012: p. 815308.

161. Anderson, M. E., Jr., et al., Patients with pediatric obstructive sleep apnea show altered T-cell populations with a dominant TH17 profile. Otolaryngol Head Neck Surg, 2014. 150(5): p. 880-6.

162. Ying, L., et al., Relationship of redundant Th17 cells and IL-17A, but not IL-17 F, with the severity of obstructive sleep apnoea/hypopnoea syndrome (OSAHS). BMC Pulm Med, 2014. 14: p. 84.

163. Toujani, S., et al., Vitamin D deficiency and interleukin-17 relationship in severe obstructive sleep apnea-hypopnea syndrome. Ann Thorac Med, 2017. 12(2): p. 107-113.

164. Minoguchi, K., et al., Increased carotid intima-media thickness and serum inflammatory markers in obstructive sleep apnea. Am J Respir Crit Care Med, 2005. 172(5): p. 625-30.

165. Can, M., et al., Effect of continuous positive airway pressure (CPAP) therapy on IL-23 in patients with obstructive sleep apnea. Immunol Res, 2016. 64(5-6): p. 1179-1184.

166. Huang, Y. S., et al., Inflammatory cytokines in pediatric obstructive sleep apnea. Medicine (Baltimore), 2016. 95(41): p. e4944.

167. Vgontzas, A. N., et al., Marked decrease in sleepiness in patients with sleep apnea by etanercept, a tumor necrosis factor-alpha antagonist. J Clin Endocrinol Metab, 2004. 89(9): p. 4409-13.

168. Bob, P., et al., Depression, traumatic stress and interleukin-6. J Affect Disord, 2010. 120(1-3): p. 231-4.

169. Cohen, M., et al., Cytokine levels as potential biomarkers for predicting the development of posttraumatic stress symptoms in casualties of accidents. Int J Psychiatry Med, 2011. 42(2): p. 117-31.

170. Hartwell, K. J., et al., Association of elevated cytokines with childhood adversity in a sample of healthy adults. J Psychiatr Res, 2013. 47(5): p. 604-10.

171. Rodney, T., et al., High IL-6 in military personnel relates to multiple traumatic brain injuries and post-traumatic stress disorder. Behav Brain Res, 2020. 392: p. 112715.

172. Lindqvist, D., et al., Interleukin-6 is elevated in the cerebrospinal fluid of suicide attempters and related to symptom severity. Biol Psychiatry, 2009. 66(3): p. 287-92.

173. Janelidze, S., et al., Cytokine levels in the blood may distinguish suicide attempters from depressed patients. Brain Behav Immun, 2011. 25(2): p. 335-9.

174. O'Donovan, A., et al., Suicidal ideation is associated with elevated inflammation in patients with major depressive disorder. Depress Anxiety, 2013. 30(4): p. 307-14.

175. Grudet, C., et al., Suicidal patients are deficient in vitamin D, associated with a pro-inflammatory status in the blood. Psychoneuroendocrinology, 2014. 50: p. 210-9.

176. Amitai, M., et al., Increased circulatory IL-6 during 8-week fluoxetine treatment is a risk factor for suicidal behaviors in youth. Brain Behav Immun, 2020. 87: p. 301-308.

177. Choi, K. W., et al., Predictive inflammatory biomarkers for change in suicidal ideation in major depressive disorder and panic disorder: A 12-week follow-up study. J Psychiatr Res, 2021. 133: p. 73-81.

1. A method of inhibiting suicidal ideations, and/or propensity towards suicide through administration umbilical cord blood mononuclear cells.

2. The method of claim 1, wherein said umbilical cord blood mononuclear cells are monocytes.

3. The method of claim 2, wherein said monocytes are M2 monocytes.

4. The method of claim 3, wherein said M2 monocytes are administered together with platelet rich plasma, wherein said cells together comprise a composition suitable for injection into an individual, and wherein said M2 monocytes are OCT-4 positive as determinable by RT-PCR, are adherent to tissue culture plastic, and are not trophoblasts.

5. The method of claim 4, wherein injection of said composition to said individual results in prolonged localization of said M2 cells at the site of injection, relative to M2 cells not combined with platelet rich plasma.

6. The method of claim 4, wherein said platelet rich plasma is autologous platelet rich plasma.

7. The method of claim 4, wherein said platelet rich plasma is derived from placental perfusate.

8. The method of claim 4, wherein the volume to volume ratio of M2 cells to platelet rich plasma in the composition is between about 10:1 and 1:10.

9. The method of claim 4, wherein the volume to volume ratio of M2 cells to platelet rich plasma in the compositions about 1:1.

10. The method of claim 4, wherein the ratio of the number of M2 cells to the number of platelets in the platelet rich plasma is between about 100:1 and 1:100.

11. The method of claim 4, wherein the ratio of the number of M2 cells to the number of platelets in the platelet rich plasma is about 1:1.

12. The method of claim 4, wherein said M2 cells are CD49f positive.

13. The method of claim 12, wherein said M2 cells are CD105 positive.

14. The method of claim 12, wherein said M2 cells are CD200 positive.

15. The method of claim 12, wherein said M2 cells are VEGFR1/Flt-1 positive.

16. The method of claim 12, wherein said M2 cells are VEGFR2/KDR positive.

17. The method of claim 12, wherein said M2 cells are CD90 positive.

18. The method of claim 12, wherein said M2 cells are OCT4 positive, HLA-G positive, and CD90 positive.

19. The method of claim 12, wherein said M2 cells are CD9 positive.

20. The method of claim 12, wherein said M2 cells are CD98 positive, CD56 positive and Tie2 positive.
